(19) United States
(12) Patent Application Publication
(10) Pub. No.: US 2021/0259902 A1
(43) Pub. Date: Aug. 26, 2021

(54) DEVICE FOR AIDING PLANTAR FLEXOR MUSCLES

(71) Applicant: Abraham Israel Luis Pena, Lima (PE)

(72) Inventor: Abraham Israel Luis Pena, Lima (PE)

(21) Appl. No.: 16/958,834

(22) PCT Filed: Dec. 26, 2018
(86) PCT No.: PCT/PE2018/000037

§ 371 Date: May 3, 2021

(30) Foreign Application Priority Data
Dec. 29, 2017 (PE) 002879-2017/DIN
Oct. 29, 2018 (PE) 002261-2018/DIN

(51) Int. Cl. A61H 3/00; A61H 1/02; B25J 9/00

(52) U.S. Cl. CPC A61H 3/00; A61H 1/0266; B25J 9/0006; A61H 2201/5069; A61H 2201/123; A61H 2201/1642; A61H 2201/165; A61H 2003/007

(57) ABSTRACT

The plantar flexure muscles assist device includes at least one drive mechanism (10) having a linear drive actuator (26); a latch mechanism (25) having a toothed annular surface coupled to the linear drive; a retractable rotating element having a toothed drum (24) and an elastic spiral element (23). The toothed drum has an annular groove and where the toothed drum corresponds to the toothed lock mechanism; a rope (28) wound in the annular groove of the toothed drum with one end attached to the elastic element (2); and a casing (21, 27) for containing the elements of the drive mechanism; a control system to send a signal to the linear actuator to activate or deactivate the locking mechanism, and a battery to supply power to the control system. The lightweight activation mechanism, together with the lightweight structure, allows the elastic element to store mechanical energy while the dorsiflexion movement occurs. This energy will enhance the plantar flexion movement, to increase physical performance on long and demanding walks and decrease user fatigue.

### CROSS REFERENCE TO RELATED APPLICATION

This application is a national stage entry of PCT/EP2018/000037 filed Dec. 26, 2018, under the International Convention and claiming priority over Peru Patent Application No. 002879-2017/DIN filed Dec. 29, 2017 and Peru Patent Application No. 002261-2018/DIN filed Oct. 29, 2018.

### TECHNICAL FIELD

This invention belongs to the field of health sciences and ergonomics, as a device that facilitates the movement of plantar flexion and thus assists in activities such as human walking.

### STATE OF THE ART

Since the last century, robotic ankle devices have been developed for the purpose of rehabilitation or physical improvement in people with temporary or permanent dysfunctionalities, which were mainly aimed at recovering the pattern of normal gait. However, despite notable progress in performance and size-reduction, due to miniaturization of electronic components, more efficient actuators, and the development of robust control systems, such devices tend to be bulky, making it difficult to use on a daily basis. So, its applicability is restricted to controlled environments, such as health centers or laboratories, in order to monitor, assist and, in some cases, evaluate the user's improvement when using the device.

One of the main reasons why these devices are not practical to use and, therefore, have not achieved mass adoption, is that they require high mechanical power to perform the movement of the foot, a function that the user usually could not achieve without the assistance. This is generally accomplished with electric motors, which are coupled to a mechanical support structure that adheres to the leg and foot externally; however, the use of these elements entails the mobilization of additional mass during the walk, which ends up penalizing the assistance. This implies a considerable disadvantage towards its daily use.

On the other hand, because its main use has resided in the clinical health area, the development of these technologies was aimed at being highly personalized, and for this reason, the physical characteristics of the ankle devices cater to the user's individual characteristics, such as its anthropometry, dysfunctionality and even the design of its use and level of assistance desired. In this sense, its design and manufacturing are generally complex, which means that its development has been particular, in order to achieve adequate assistance. Also, most of these devices restrict the movement of the ankle joint, which has two degrees of freedom, to only one in the sagittal plane, in order to stabilize it. Although this is desired for a user with dysfunctional gait, it is uncomfortable for a user who does not present it, because it restricts natural movement.

Thus, for example, it is proposed to apply ankle assistive devices to enhance and protect the joint in order to increase physical performance on long and demanding walks, which would be valuable to increase productivity in sectors such as mining or construction; likewise, greatly reducing fatigue from long walks.

On the other hand, active systems provide assistance through electric, pneumatic or hydraulic actuators. These systems tend to be heavy, due to the metallic components they carry, in addition to the battery they require, which has a weight proportional to its operating time. Likewise, they do not solve the technical problems of free movement of the ankle, that is, they do not restrict any of its two degrees of freedom, have easy portability, and support to the body, and possess a light and highly efficient drive mechanism. Examples of this type of system are the patent “Active ankle and foot orthosis” with patent code U.S. Pat. No. 8,075 633B2 and “Rigid ankle and foot orthosis” with patent code: U.S. Pat. No. 6,689,081 B2, which uses an electric motor to support the movement of the foot.

Both types of systems, passive and active, have shown effective assistance in supporting gait movement. However, they also have relevant limitations that have not yet been overcome. The main one is that they do not include a simple drive mechanism, where the driven principle does not depend on a motor, but on an elastic element, capable of storing energy in the dorsiflexion movement and enhancing the plantar flexion movement, without depending on rigid structures made of heavy materials, likewise, these structures only allow the movement of the foot in a degree of freedom, limiting the pronation and supination movements of the foot.

That is why one of the most recent inventions, called “Assistive Device for Plantar Flexion Muscles” presented in Peru with file number 002824, proposed an articulated structure with two degrees of freedom, which improves portability. In addition, it uses a quasi-passive drive system, which implies that it is electronically controlled, requiring the need for a battery, but the driven principle does not depend on a motor, but on an elastic element, so the mechanical power requirements for the assistance are less, which implies a reduction in the mass of the drive system, and therefore in the assistance device itself.

### DESCRIPTION OF THE INVENTION

The present invention solves the aforementioned problems of the state of the art, proposing a new assistance system for the movement of plantar flexion muscles that employs a quasi-passive drive system, which is worn in the textile form as much as possible, with the minimum of rigid structures and heavy material. This reduces the weight of said equipment and therefore improves its portability and efficiency of assistance. Likewise, the driving principle does not depend on a motor, but on an elastic element, where the proposed drive mechanism hooks and disengages an elastic element through elements that comprise an actuator with a linear drive that displaces a toothed lock type mechanism, joining it with a retractable rotating element and thus restricting its rotation. This allows the elastic element to store mechanical energy, while the dorsal flexion movement occurs. Later, said energy will enhance the plantar flexion movement, in order to increase physical performance in long and demanding walks and decrease user fatigue.

The present invention intends to imitate the elastic behavior of tendons, through energy storage, by means of an external elastic element in order to increase the efficiency of plantar flexion movement.

The present invention comprises a support clamp that covers the user's calf, a foot clamp that covers the user's footwear, and is located at the bottom with respect to the support clamp and at least one drive mechanism that is mounted on the anterior part of the support clamp, where the drive mechanism is attached to the support clamp and the foot clamp through an elastic element characterized in that:

the at least one actuating mechanism comprises an actuator with a linear actuation; a latch type mechanism having a toothed annular surface coupled to the linear drive; a retractable rotating element composed of a toothed drum and an elastic spiral element; where the toothed drum has an annular groove and where the toothed drum corresponds to the toothed lock type mechanism; a rope wound in the annular groove of the toothed drum with one end attached to the elastic element; and a casing to contain the elements of the drive mechanism;

the support clamp comprises at least one adjustable strap that embraces the front part of a calf transversely and supports the drive mechanism at the rear part of the calf; preferably, the support clamp comprises three adjustable straps made up of an upper strap that is located between the knee and the proximal part of the calf muscle; a mid-strap which wraps a wider section than the top strap due to the geometry of the calf muscle; and a lower strap which embraces the distal part of the calf. This configuration has the purpose of fixing the calf support and also adapts to different anthropometries of the legs within a normal population range.

The foot clamp comprises a foot clamp structure, which preferably has a “U” shape, with support sole for the back and bottom part of the shoe, which embraces and is fixed to a part of a shoe externally, in order to be attached to conventional footwear and where the foot clamp has protrusion on its back where the elastic element is placed. Likewise, in a preferred form, the foot clamp has an adjustable instep strap that is located in the front part of the foot clamp structure, the support sole could be an “L” shaped plate suitable to be located in the bottom and front of the shoe and support the weight of the person. The instep strap is located in the front part of the foot clamp structure and can be inclined, so as to be parallel to the instep of the user's shoe: the sole on its back can comprise a sole or cross plate which connects to the sides of the foot clamp structure at the height of the sides of the user's footwear.

In another preferred embodiment, the drive mechanism has a fixed shaft and shaft coupling which together form an axis of rotation for the retractable rotary element. The elastic element that connects the support clamp and the foot clamp, if it is metallic, can have an anticorrosive coating to increase its durability.

The back of the bracket support could be rigid for better support of the drive mechanism.

The drive mechanism is located in the support clamp and has the ability to engage, when activated, and disengaged when deactivated. The support clamp to the foot clamp, through an elastic element, is achieved with the components that make it up. To activate its operation, electronic components are used, which monitor the movement of the leg and send a signal to control its operation.

The materials in the manufacture of the elements of the aforementioned subsystems must be light and dimensioned for the load they bear.

On the other hand, the foot clamp is composed of a structure that has the function to wrap the footwear and, through elements called the soles and the adjustable straps on the instep, to attach it to the foot. The foot clamp can have a substantially U-shaped or ellipsoidal part with a straight part in the lateral or extreme areas, which allows it to be fitted in conventional footwear, and it also has protrusion on the back. In said protrusion, the elastic element is placed as part of the actuation mechanism, located in the support bracket. Thus, if the actuation mechanism is activated and therefore hooked, and the angle of the ankle comprised by the relative position of the foot with respect to the leg decreases, then the elastic element will begin to stretch, which will pull the foot of the person through the foot clamp fixed to this segment. This pull will manifest as a pushing force on the sole of the foot. For what has been previously described, it is specified that the foot clamp has two fundamental characteristics: in the first instance, it can be dressed externally to the footwear, even without the need to remove it. Therefore, it is easy to install on the user; and secondly, it has high efficiency of assistance due to the fact that the pushing force is developed throughout the sole, especially in the heel, which increases the lever movement generated by the action of the force of the elastic element of the drive mechanism located in the support bracket.

The operating principle of the drive mechanism is supported by a control system implemented in an electrical circuit, as follows: when the actuator is activated, the locking mechanism moves, which blocks the movement of the retractable rotating element, which is connected to a rope with an elastic element. Therefore, by blocking the movement of the retractable rotary element, and the rope being tensioned, the elastic element begins to store energy if the rope is pulled. This occurs naturally in plantar flexion movements, such as in the human walk: if the user wears the device, at the beginning of the walk when stepping, the angle of the ankle decreases, and consequently, the relative distance between the support clamp located on the leg and the protrusion of the back of the foot clamp increases. Therefore, the rope will pull the elastic element, which will begin to store energy due to stretching until the ankle angle stops decreasing: also, this energy will be released when the angle increases, which happens in the walk in the movement called propulsion. This sequence will be carried out in the support phase during the walk, that is, when the foot is in contact with the floor. On the other hand, during the balancing phase, that is, when the foot is not in contact with the floor, the drive system will not activate.

In this sense, the control system, implemented in electronics, which is positioned at the back of the support bracket, preferably in a rigid part of said bracket, will allow the drive mechanism to be controlled, in such a way that it allows storing energy through an elastic element, and use this stored energy to enhance the movement of the ankle. The control system will monitor the movement of the user during the march in order to detect two events: contact and take-off of the sole of the foot, to estimate the moments of assistance. Then, the control system comprising at least one sensor configured to detect the movement of the user's walk, a microcontroller configured to receive the data from at least one sensor and send a signal to the actuator with linear actuation to activate or deactivate the mechanism lock type and a battery to supply power to the control system.

Therefore, the present invention intends to imitate the elastic behavior of the tendons, through energy storage by means of an external elastic element in order to increase the efficiency of the plantar flexion movement. Likewise, since it does not have mostly rigid elements that connect the various parts of the structure (as other inventions in the state of the art have developed), it allows free movement of pronation and supination in the frontal plane and dorsal flexion and on the sagittal plane, both without restriction. Also, the drive mechanism of the present invention, as well as the operating principle of the clamps, could be used to assist other joints of the body, such as the arm, knee, and man.

### BRIEF DESCRIPTION OF THE FIGURES

To complete the description that is being carried out and in order to facilitate the understanding of the characteristics of the invention, a set of figures is attached to the present specification in which, by way of illustration and not limitation, the following has been represented.

FIG. 1: Front view of the invention, showing how the device is dressed.

FIG. 2: Exploded view, which shows the support clamp and the elements it contains.

FIG. 3: Isometric view, which shows the support clamp and its fastening elements.

FIG. 4: Exploded view, which shows the parts of the foot clamp.

FIG. 5: Exploded view, which shows the drive mechanism and its components.

FIG. 6: Front view of a preferred embodiment, showing the bar and the upper support.

FIG. 7: Exploded view, which shows how the bar and the foot clamp are joined.

FIG. 8: Exploded view showing how the bar, the rigid part of the support bracket, and the drive mechanism are joined.

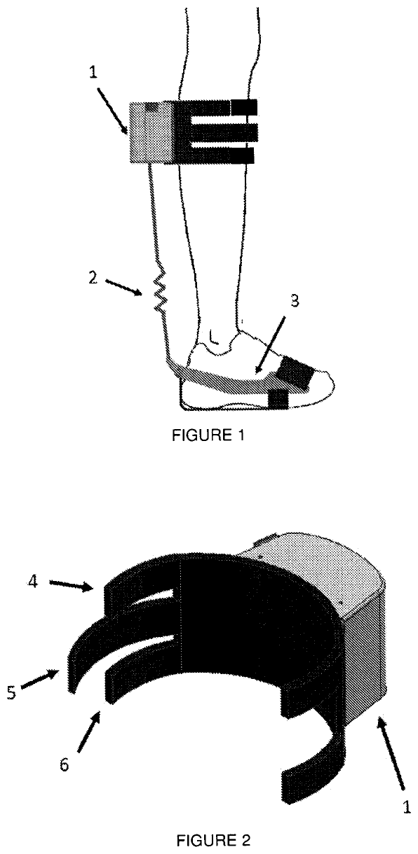

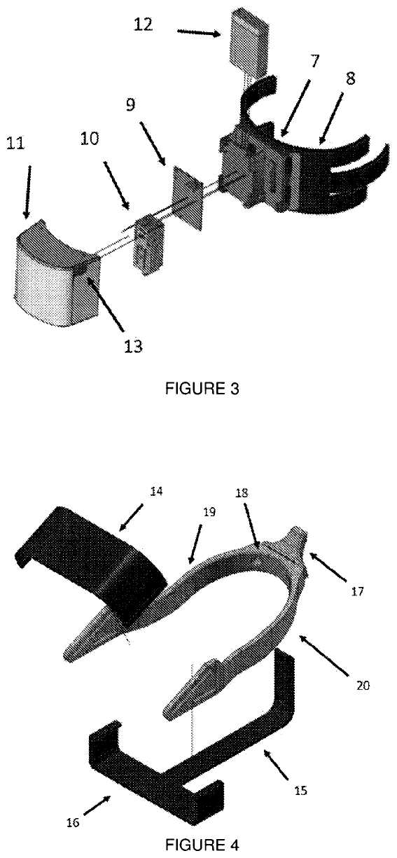

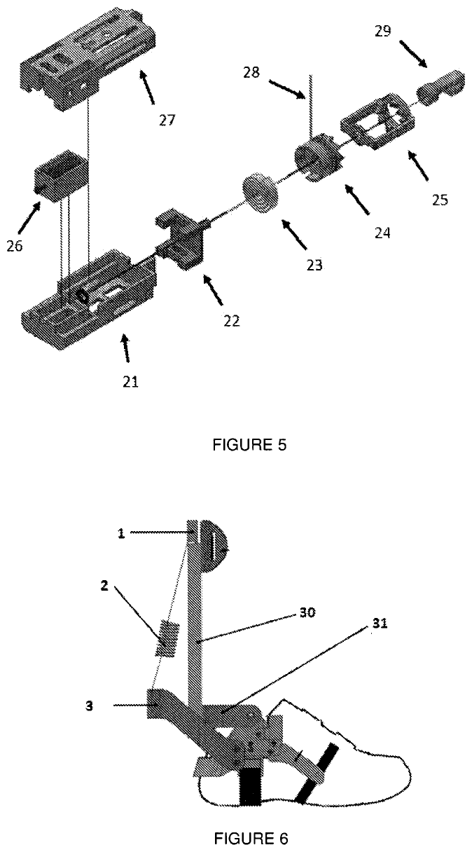

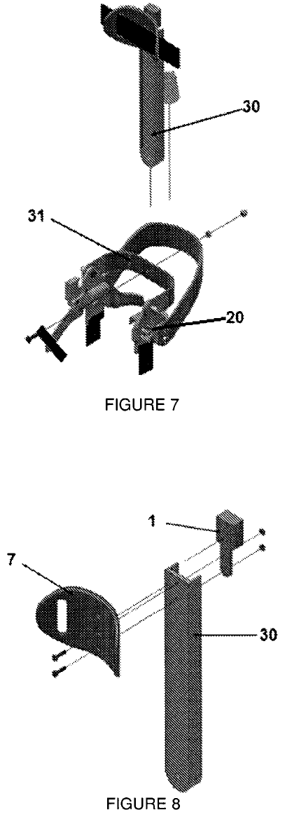

### PREFERRED EMBODIMENT OF THE INVENTION

The present invention comprises a support clamp (1), a foot clamp (3), and at least one actuation mechanism (10) which joins the foot clamp and the foot clamp through an elastic element (2).

The support clamp (1), which is conceived as a calf coupling with improved clamping characteristics, can be made up of a rigid part (7), preferably constituted with a polymeric material, such as plastic or light metal, such as aluminum, which has an anatomical shape and ellipsoidal section in such a way that it is coupled to the anterior part of the calf, and a flexible part (8) made up of at least three adjustable straps located at both lateral ends of the rigid part (7), which start from there and wrap the front of the calf transversely. Also, the flexible part is made up of an upper strap (4) that is located between the knee and the proximal part of the calf muscle and is made of a material that is foldable but not elastic or deformable in length, such as a textile tape to use Velcro on the ends of these straps to fix them; a middle strap (5) which embraces a wider section than the aforementioned due to the geometry of the calf muscle and can be made of a rigid or flexible material, such as, for example, a strap made of textile or elastic material; and a lower strap (6) which embraces the distal part of the calf and is made of a material that is foldable, but not elastic or deformable in length, such as a textile material tape that has Velcro at the ends of the tape in such a way that can be fixed, one with respect to the other.

The foot clamp (3) is composed of a foot clamp structure (20) which embraces the shoe externally, has a substantially “U” part or ellipsoidal section (18) at the back and a straight part (19) on the side parts with a length that is approximately half that of the footwear in which it is to be worn. This allows it to be attached to conventional footwear. Also, the foot clamp structure has the protrusion on the back (17). In the protrusion (17), the elastic element (2) is placed, which is attached to the actuation mechanism (10) that is located in the support clamp. The foot clamp structure (20) is attached to the shoe at the back and bottom through the sole or cross plate (16) and the support sole (15), and the instep straps (14). The transverse sole (15) is fixed on both lateral sides of the rigid part (19) of the foot clamp structure (3), in the section in which the rigid part ends. Likewise, the support sole (15), preferably made with an “L” shape, is located parallel to the front/back of the shoe, starting from the protrusion (17) and until reaching the sole or transverse plate (16). Likewise, the sole or transverse plate and the support sole are preferably made of thin material, resistant to wear and tear because they would be stepped on by the user when using the device, such as thin leather or thin ribbons or high resistance/durability cords, or even its elaboration material or design could be modified according to the aesthetic style, or technical needs of the terrain. On the other hand, the instep straps (14) are located at the front of the foot clamp structure (20) at a certain angle, such that it is located on the user's instep. The instep straps are made of rigid material, such as plastic or flexible-like tapes.

The drive mechanism (10) comprises a linear drive actuator (26), a latch type mechanism (25) coupled to the linear drive having a toothed surface, a retractable rotating element composed of a toothed drum (24) and a spiral element elastic (23), a rope (28) wound in an annular groove or slot of the toothed drum with one end attached to an elastic element (2), and a protective casing, which can be divided into two parts (21, 27). The elastic element allows energy to be stored depending on the activation of the drive mechanism (10) through a control system, which is implemented in an electronic circuit (9) which comprises a microcontroller and at least one sensor that measures the angle and angular velocity of the foot, with respect to the horizontal plane during the walking phase, powered by a battery (12). The mechanism allows the energy storage in the elastic element (2) to be controlled as follows: When the ankle's angle between the user's leg and foot is reduced, the control system detects this movement and sends a signal to the actuator with linear drive. When activated, it displaces the locking mechanism, which blocks the movement of the retractable rotating element. This causes the elastic element (2) to stretch when the angle is shortened, which stores energy in said element. This energy will increase as the angle is shortened, and will deliver when the angle is enlarged. Also, the linear drive (26) is preferably an electric actuator; however, it can also be pneumatic, although it should include all the necessary components for its implementation. Preferably, the drive mechanism has a fixed shaft (22) and a shaft coupling (29) that together form an axis of rotation of the retractable rotary element.

Likewise, the operation of the drive mechanism to support normal walking is as follows: When the control system detects that the foot initiates contact with the floor, it sends a signal to the linear drive actuator (26), which moves the mechanism type lock (25), and thus blocks the movement of the retractable rotating element. Said retractable rotating element is blocked and, therefore, when the ankle angle decreases, which happens after the foot is in contact, the elastic element (2) will stretch, which will store elastic energy. In the walk, the angle is shortened during the initial part of the support phase. Subsequently, at the end of the support phase, the foot comes off the ground, causing the angle of the ankle to enlarge. This movement is called propulsion. Thus, during this stage, the energy stored in the elastic element will be used to support the foot's detachment from the ground. This force will help facilitate the walk during the propulsion phase. After the support phase is the balance phase, in which the foot is not in contact with the floor. So, the drive mechanism will remain off, not storing energy.

The electronic components will preferably be light and small. For monitoring and detecting movement on the walk, sensors such as accelerometers, gyros, inertial sensors, or a combination of the above could be used. This is done to predict the movement of the user and adequately assist the movement. On the other hand, the battery (12) will preferably be lithium-ion or another light battery with high storage capacity. Also, the control would use electronic controllers such as embedded microcontrollers.

In other words, the control system sends the activation signal to start storing energy in the drive mechanism that occurs when the foot performs the dorsiflexion movement, which occurs when the entire sole of the foot is in contact with the floor during the support phase of the march. After reaching the minimum dorsal flexion angle, the plantar flexion movement begins, in which the previously stretched elastic element (2) begins to contract to return to its original position. Said effort of the elastic element provides an additional impulse to that of the muscle, therefore, assists the foot to make the propulsion movement and, therefore, allows the plantar flexion movement to be carried out with less effort. The control system sends the deactivation signal when the balance phase begins, that is, the foot is no longer in contact with the floor.

The operation of the said drive mechanism is clear to visualize in the walk due to the cyclical movements of dorsal and plantar flexion; however, it could also promote other activities such as running or jumping. Therefore, the proposed invention would not only assist the plantar flexion movement during the walk, but also others, and as long as a dorsiflexion movement precedes, it allows energy to be stored in the elastic element of the drive mechanism.

Furthermore, the drive mechanism (1) of the present invention could be used to assist other joints of the body, such as the arm, knee, or shoulder.

Finally, the elements of the previously described subsystems must be manufactured with a light material and dimensioned according to the loads they will bear. For example, it is proposed that the rigid structures that make up the device be made of an aluminum alloy, carbon fiber, or some highly resistant low-density composite material. On the other hand, the fastening elements such as the upper strap, middle strap and lower strap, as well as the instep straps, are made of a lightweight material that is resistant to corrosion such as high temperatures or water. In addition, they must be soft in order to provide comfort when in contact with the user. Taking into account the foregoing, the present invention weighs a total of between 300 and 500 g, a weight much less than the devices of the state of the art.

1. A muscle assist device comprising:
a support clamp (1),
a foot clamp (3), and
at least one actuation mechanism (10) which attaches the support clamp and the foot clamp through an elastic element (2);
wherein the at least one actuation mechanism (10) comprises an actuator with a linear actuation (26); a latch mechanism (25) having a toothed annular surface coupled to the linear actuation; a retractable rotating element composed of a toothed drum (24) and an elastic spiral element (23);
wherein the toothed drum has an annular groove and the toothed drum corresponds to the toothed lock type mechanism; a rope (28) wound in the annular groove of the toothed drum with one end attached to the elastic element (2); and a casing (21, 27) for containing the elements of the drive mechanism; a control system comprising at least one sensor configured to detect the movement of the user's walk, a microcontroller configured to receive data from the at least one sensor and send a signal to the actuator with a linear drive to activate or deactivate the locking mechanism, and a battery to supply power to the control system. The support clamp (1) comprises at least an adjustable strap that embraces the front part of a calf in a transversal manner and supports the actuation mechanism in the rear part of the calf. The foot clamp (3) comprises a foot clamp structure (20) connected with a support sole (15) for the rear and lower part of the shoe that embraces and is fixed to part of a shoe externally, in order to be attached to conventional footwear and where the foot clamp structure has a protrusion (17) on its back where the elastic element (2) is placed.

2. The assistive device for plantar flexion muscles according to claim 1, wherein the least one adjustable instep strap (14), that is located in the anterior part of the foot clamp structure (20).

3. The assistive device for plantar flexion muscles according to claim 2, wherein the foot clamp structure (20) is “U” shaped.

4. The assistive device for plantar flexion muscles, according to claim 1, wherein the actuation mechanism has a fixed axis (22) and a shaft coupling (29) that together form an axis of rotation of the element retractable swivel.

5. The assistive device for plantar flexion muscles, according to claim 1, wherein the support bracket (1) comprises three adjustable straps composed of an upper strap (4) that is located between the knee and the proximal part of the calf muscle; a mid strap (5) which wraps a wider section than the top strap due to the geometry of the calf muscle; and a lower strap (6) which embraces the distal part of the calf.

6. The assistive device for plantar flexion muscles: according to claim 1, wherein the elastic element (2) has an anticorrosive coating.

7. The assistive device for plantar flexure muscles according to claim 2, wherein the instep strap (14) is located in the front part of the foot clamp structure (20) inclined in order to be located parallel to the instep of the user's footwear.

8. The assistive device for plantar flexure muscles, according to claim 2, wherein the sole (15) is connected to the foot clamp structure (20) by its rear part; and the sole (15) in its front part comprises a transverse plate (16) which connects to the sides of the foot clamp structure (20) at the same height as the sides of the user's foot.

9. The assistive device for plantar flexure muscles, according to claim 3, wherein the support clamp (1) comprises a bar that is joined to a “U” shaped upper support at its rear through a pivoting element, whose axis of the pivoting element is perpendicular to the frontal anatomical plane, which allows the user's ankle to make pronation and supination movements, the ends of the upper “U” shaped support join with the ends of the structure foot clamp (20) through two pivoting elements perpendicular to the sagittal anatomical plane, which allows the user's ankle to perform the movements of plantar flexion and dorsal flexion.

10. An actuation mechanism for plantar flexure muscles assist device comprising:
a linear actuator (26);
a latch mechanism (25) having a toothed annular surface coupled to the linear drive;
a retractable rotating element composed of a toothed drum (24); and
an elastic spiral element (23);
wherein the toothed drum has an annular groove and where the toothed drum meshes with the toothed lock mechanism; a rope (28) wound in the annular groove of the toothed drum with one end attached to an elastic element (2); and a casing (21, 27) for containing the elements of the drive mechanism.
